(19) United States
(12) Patent Application Publication
(10) Pub. No.: US 2022/0041833 A1
(43) Pub. Date: Feb. 10, 2022

(54) Membranes Suitable for Detecting, Filtering and/or Purifying Biomolecules

(71) Applicant: Fujifilm Manufacturing Europe B.V., Tilburg (NL)

(72) Inventors: Yujiro Itami, Tilburg (NL); Berry Bogels, Tilburg (NL)

(21) Appl. No.: 17/276,615

(22) PCT Filed: Sep. 4, 2019
(86) PCT No.: PCT/GB2019/052458

§ 371 Date: Mar. 16, 2021

(30) Foreign Application Priority Data
Sep. 21, 2018 (GB) 1815405.4

(51) Int. Cl. C08J 9/28; C08F 220/60; C07K 1/18; B01D 69/02; B01D 69/12; B01D 67/00; B01D 71/56

(52) U.S. Cl. CPC C08J 9/286; B01D 15/34; C07K 1/18; B01D 69/02; B01D 69/125; B01D 67/0006; B01D 67/0009; B01D 71/56; C08F 2810/20; C08J 2333/26; C08J 2205/042; C08J 2205/044; B01D 2325/02; B01D 2325/16; B01D 2323/30; B01D 2323/345; C08F 220/60

(57) ABSTRACT

Membranes having an average pore size of 5 nm to 5,000 nm and a porosity of 15% or more, said membrane being obtainable by a process comprising curing a composition comprising:
5 to 64 wt % of (i) a cross-linking agent comprising at least one cationic group; and
36 to 95 wt % of (ii) inert solvent(s).

The membranes are useful for detecting, filtering and/or purifying biomolecules.

The present invention relates to membranes and to their preparation and use, e.g. for detecting, filtering and/or purifying biomolecules.

A number of techniques are known for the detection, filtration and purification of biomolecules (e.g. proteins, amino acids, nucleic acids, anti-bodies and endotoxins). These techniques include size-exclusion chromatography where biomolecules are separated and/or purified based on their size (i.e., physical exclusion)) and in ion exchange chromatography where biomolecules are purified or separated according to the strength of their overall ionic interaction with ionic groups in a membrane.

The present invention sets out to provide membranes which have good tolerance to high and low pH and can be used for the detection, filtration and purification of biomolecules, especially membranes which are porous and have a good ion exchange capacity and water flux.

According to a first aspect of the invention there is provided a membrane having an average pore size of 5 nm to 5,000 nm and a porosity of 15% or more, said membrane being obtainable by a process comprising curing a composition comprising:
5 to 64 wt % of (i) a cross-linking agent comprising at least one cationic group; and
36 to 95 wt % of (ii) inert solvent(s).

### BRIEF DESCRIPTION OF THE DRAWINGS

FIGS. 1a and 1b are scanning electron microscope (“SEM”) photographs of a cross-section through a membrane according to the present invention at a magnification of ×10,000. The membrane in FIGS. 1a and 1b does not comprise a porous support.

FIG. 2a is an SEM photograph of the surface of a membrane according to the present invention at a magnification of ×2,000. FIGS. 2b and 2c are SEM photographs of a cross-section through a membrane according to the present invention at a magnification of ×400 and ×2,000 respectively. The membrane in FIGS. 2a, 2b and 2c comprises a porous support.

FIG. 3a is an SEM photograph of the surface of a further membrane according to the present invention at a magnification of ×2,000. FIGS. 3b and 3c are SEM photographs of a cross-section through a membrane according to the present invention at a magnification of ×400 and ×2,000 respectively. The membrane in FIGS. 3a, 3b and 3c comprises a porous support.

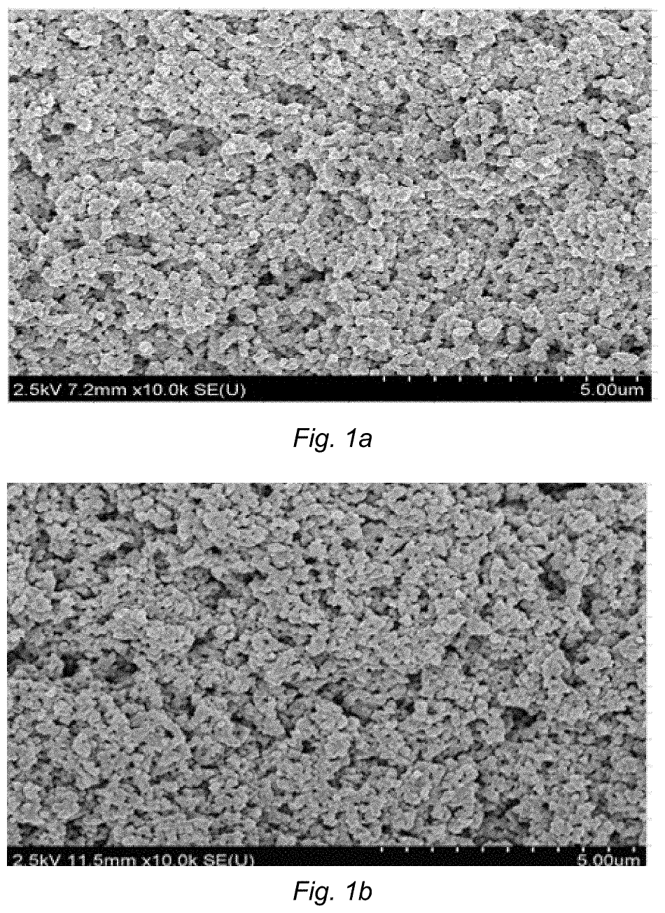

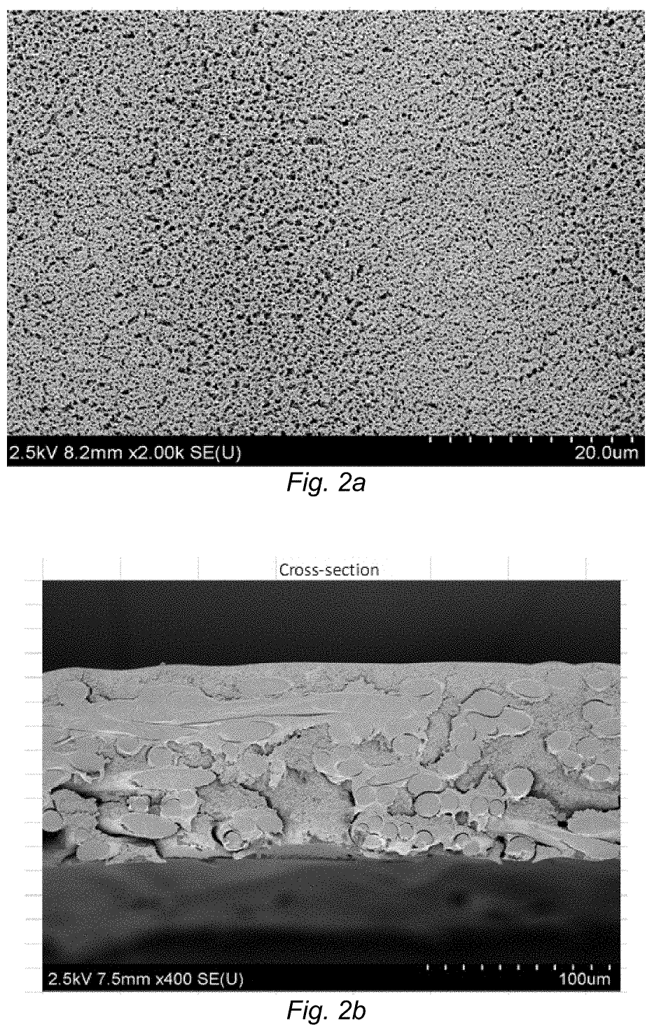

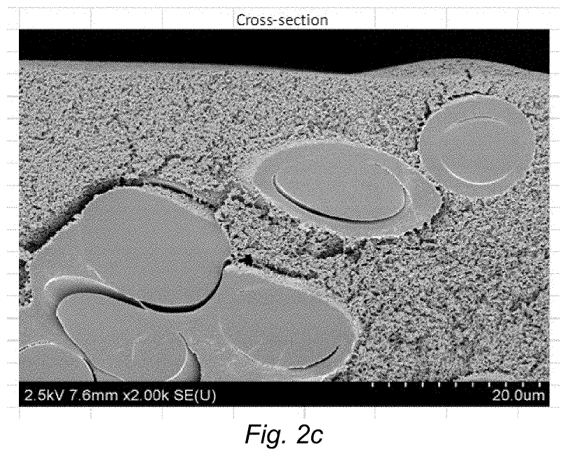

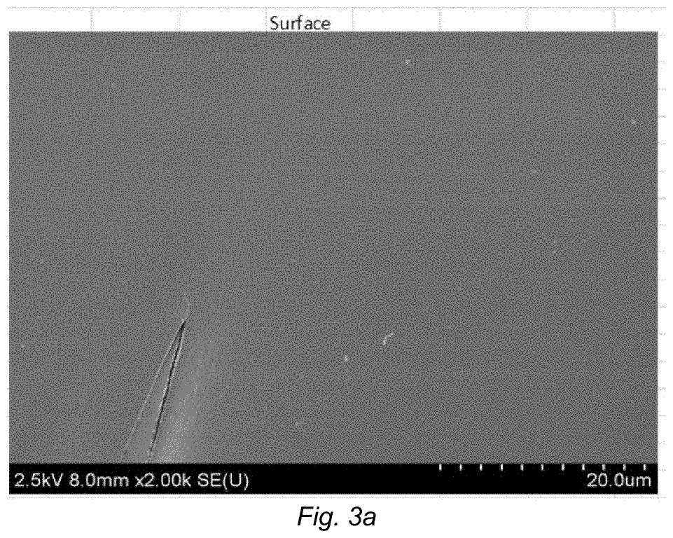

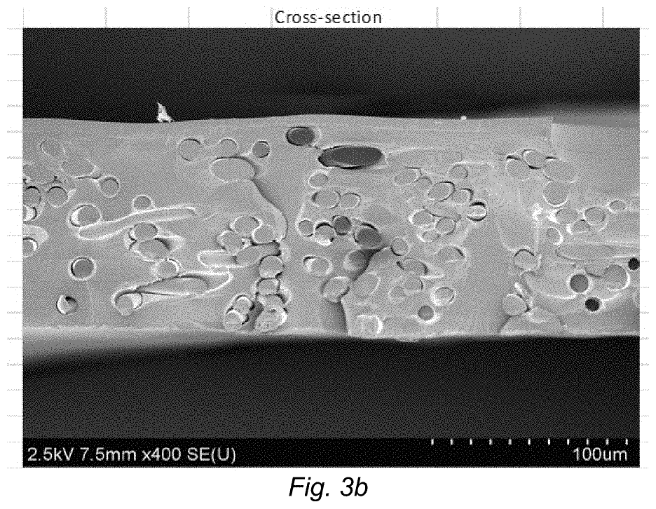

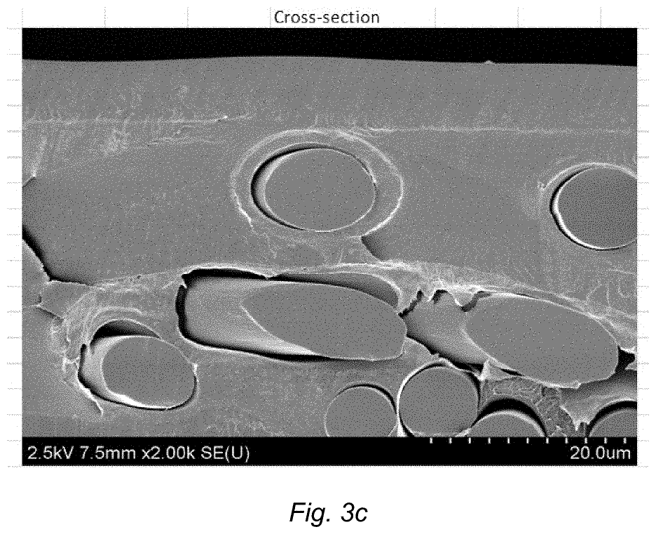

In this specification (including its claims), the verb “comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The cross-linking agent preferably comprises at least two polymerisable groups, e.g. at least two groups selected from epoxy, thiol (—SH), oxetane and especially ethylenically unsaturated groups. The polymerisable groups in component (i) will typically be selected such they are reactive with each other or with at least one polymerisable group present in another, chemically different component (i).

Curing causes the cross-linking agent to cross-link, e.g. to form the membrane as a crosslinked, three dimensional polymer matrix. Thus the cross-linking agent can be contrasted with substances such as diallyldimethylammonium chloride which are not cross-linking agents and instead form a linear homopolymer.

The at least two polymerisable groups present in component (i) may all be chemically identical or they may be different. The at least two polymerisable groups present in component (i) are cross-linkable.

Preferred ethylenically unsaturated groups are selected from (meth)acrylic groups and vinyl groups (e.g. vinyl ether groups, aromatic vinyl compounds, N-vinyl compounds and allyl groups).

Examples of suitable (meth)acrylic groups include acrylate (H₂C═CHCO—) groups, acrylamide (H₂C═CHCONH—) groups, methacrylate (H₂C═C(CH₃)CO—) groups and methacrylamide (H₂C═C(CH₃)CONH—) groups. Acrylic groups are preferred over methacrylic groups because acrylic groups are more reactive.

Preferred ethylenically unsaturated groups are free from ester groups because this can improve the stability and the pH tolerance of the resultant membrane. Ethylenically unsaturated groups which are free from ester groups include (meth)acrylamide groups and vinyl ether groups ((meth)acrylamide groups are especially preferred).

As preferred examples of polymerisable groups there may be mentioned groups of the following formulae:

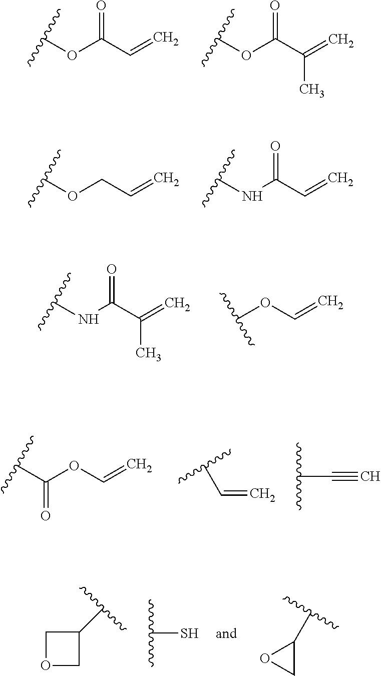

The cationic group(s) in component (i) can help the resultant membrane to distinguish between ionic species such as ionically charged biomolecules. Preferred cationic groups include quaternary ammonium groups and quaternary phosphonium groups.

In a preferred embodiment component (i) comprises at least two cationic groups, e.g. two, three or four cationic groups. The cationic group(s) are preferably linked to the remainder of component (i) through a mono or divalent covalent bond.

Preferred examples of component (i) include the compounds (M1) to (M32) below:

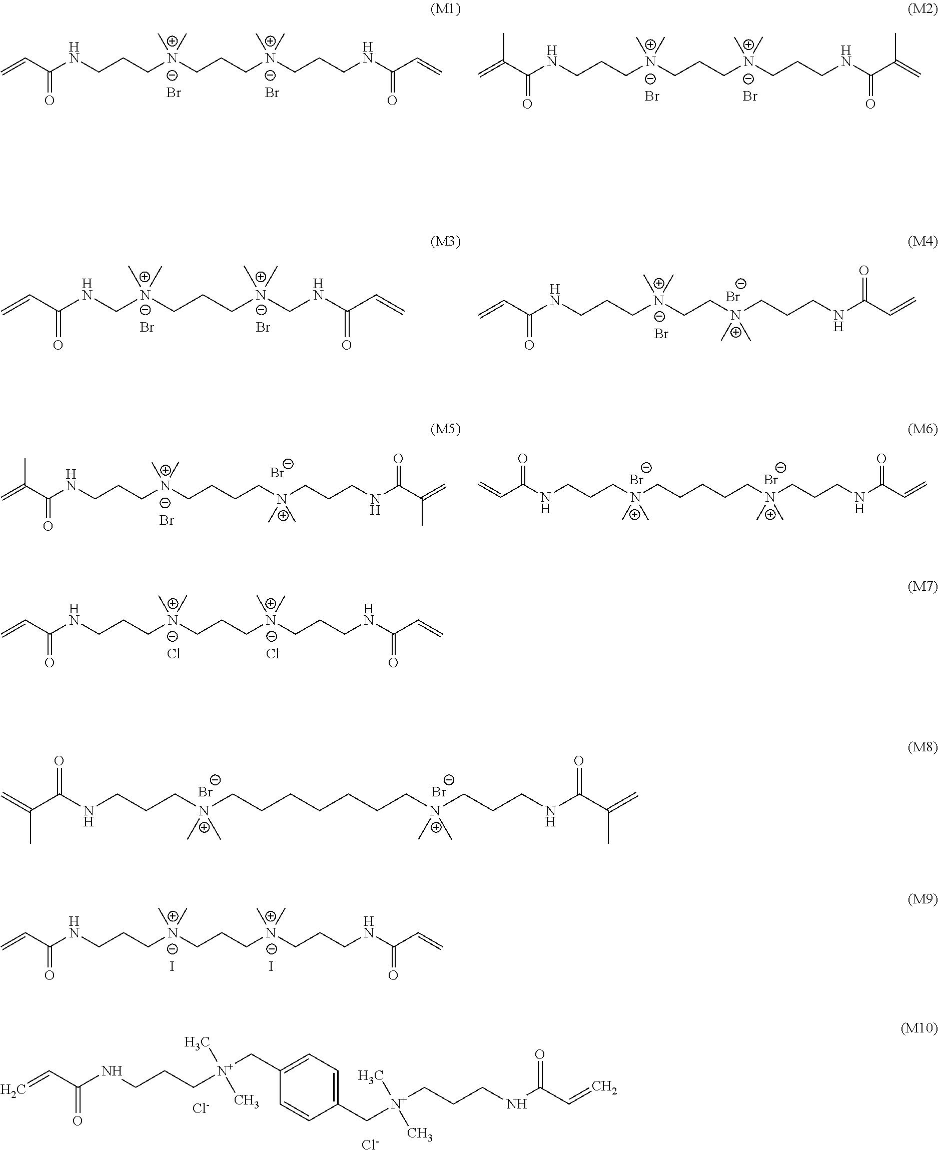

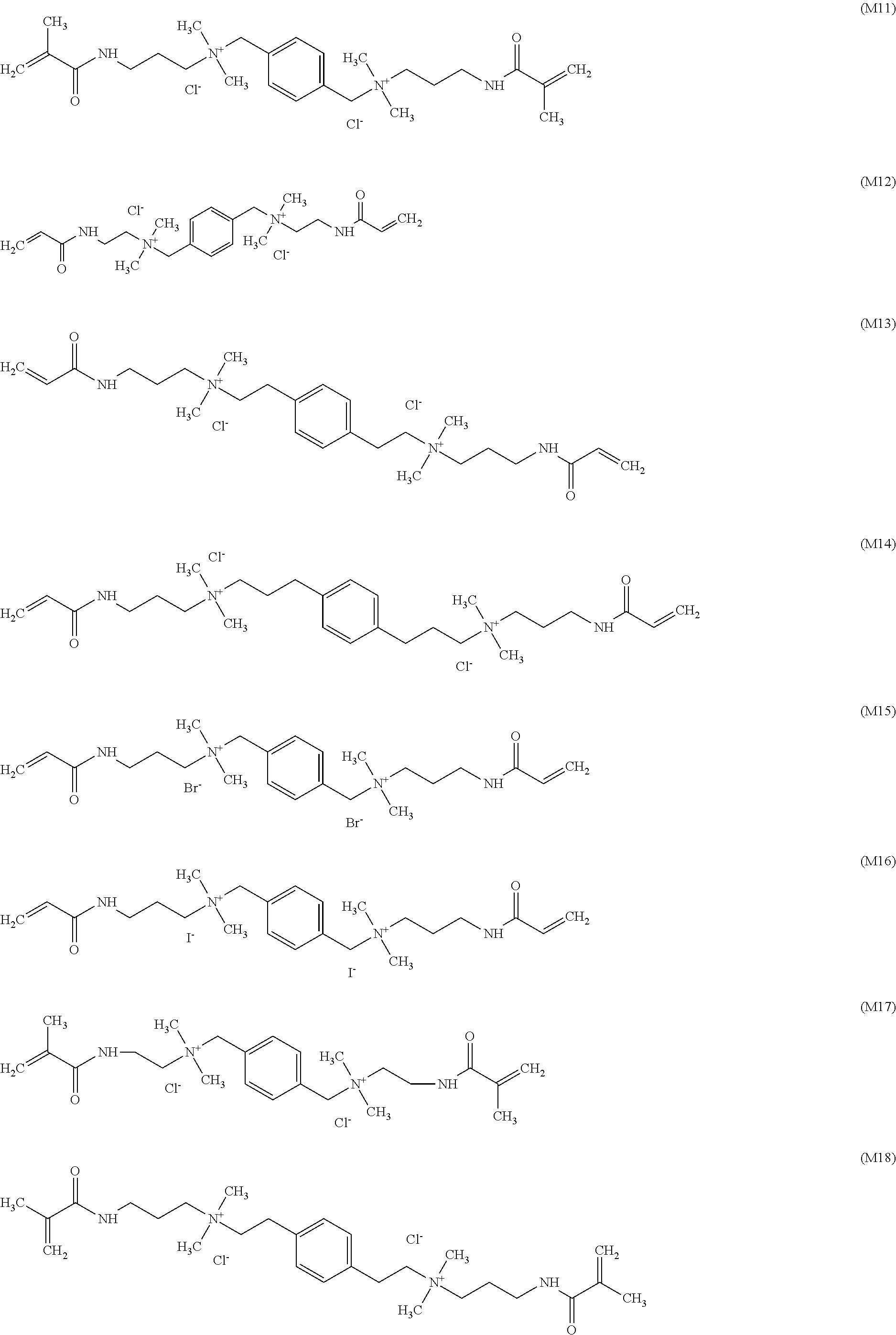

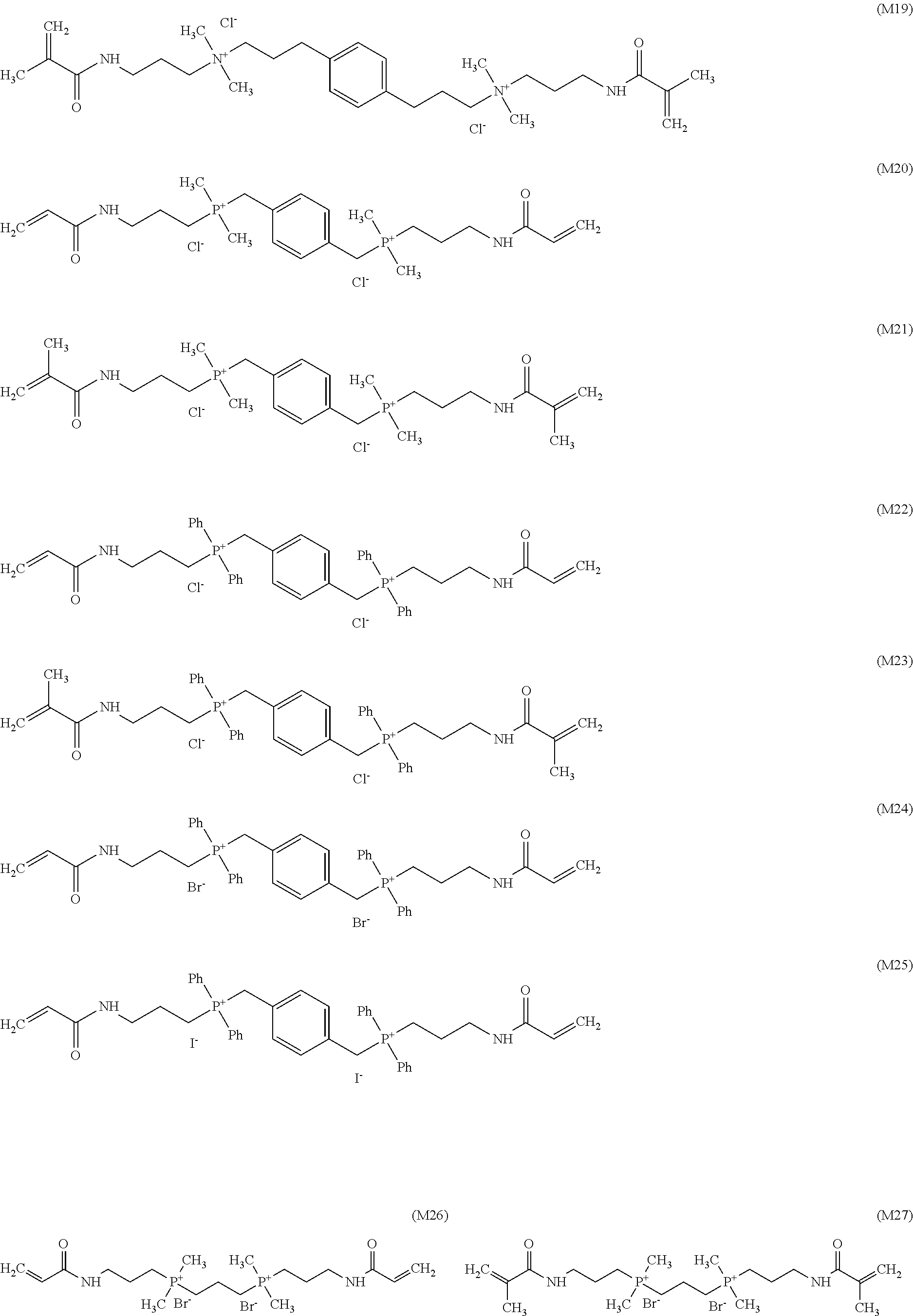

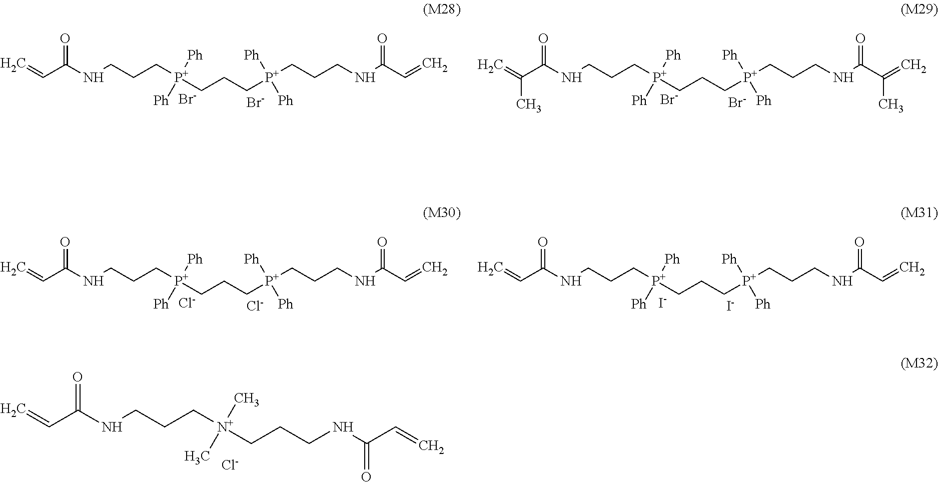

The amount of component (i) present in the composition, relative to the total weight of the composition, is preferably 10 to 64 wt %, more preferably 15 to 64 wt %, based on the total weight of the composition.

Preferably component (i) is completely dissolved in the composition.

In this specification “inert” means non-polymerisable. Thus component (ii) is incapable of polymerising with component (i).

Component (ii) preferably consists of one inert solvent or comprises more than one inert solvent, especially a mixture comprising two or more miscible inert solvents. The inert character of component (ii) assists the formation of pores in the membrane.

Preferably component (ii) is a non-solvent for the membrane (the membrane is preferably insoluble in component (ii)). Component (ii) performs the function of dissolving component (i), component (iv) and optionally component (iii) (when present). Component (ii) can also help to ensure that the membrane precipitates from the composition as it is formed, e.g. by a phase separation process.

The amount of component (ii) present in the composition, relative to the total weight of the composition, is preferably 36 to 98 wt %, more preferably 36 to 95 wt %, especially 40 to 95 wt % and more especially 45 to 95 wt %, based on the total weight of the composition.

Preferably component (ii) comprises water, or a mixture of water and an inert, organic solvent (preferably a water-miscible, inert organic solvent) having a water-solubility of at least 5 wt %.

Examples of inert solvents which may be used as or in component (ii) include alcohol-based solvents, ether-based solvents, amide-based solvents, ketone-based solvents, sulfoxide-based solvents, sulfone-based solvents, nitrile-based solvents and organic phosphorus-based solvents, of which inert, aprotic, polar solvents are preferred.

Examples of alcohol-based solvents which may be used as or in component (ii) (especially in combination with water) include methanol, ethanol, isopropanol, n-butanol, ethylene glycol, propylene glycol, diethylene glycol, dipropylene glycol and mixtures comprising two or more thereof. Isopropanol is particularly preferred.

In addition, preferred inert, organic solvents which may be used as or in component (ii) include dimethyl sulfoxide, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate, y-butyrolactone and mixtures comprising two or more thereof. Dimethyl sulfoxide, N-methyl pyrrolidone, dimethyl formamide, dimethyl imidazolidinone, sulfolane, acetone, cyclopentylmethylether, methylethylketone, acetonitrile, tetrahydrofuran, 2-methyltetrahydrofuran and mixtures comprising two or more thereof are preferable.

In a preferred embodiment component (ii) comprises a composition comprising one of inert solvents selected from list (iia) and one or more inert solvents selected from list (iib):
list (iia): iso-propanol, methanol, ethanol, acetone, tetramethyl urea, hexamethyl phosphoramide, hexamethyl phosphorotriamide, butanone, cyclohexanone, methylethylketone, tetrahydrofuran, tetrahydropyran, 2-methyltetrahydrofuran, cyclopentylmethylether, propionitrile, acetonitrile, 1,4-dioxane, 1,3-dioxolane, ethyl acetate, y-butyrolactone or a mixture comprising two or more thereof; and
list (iib): water, glycerol, ethylene glycol, dimethyl sulfoxide, sulpholane, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate and y-butyrolactone, and among these, dimethyl sulfoxide, N-methyl pyrrolidone, N,N-dimethyl formamide, dimethyl imidazolidinone, N-methyl morpholine, acetone, cyclopentylmethylether, methylethylketone, acetonitrile, tetrahydrofuran, 2-methyltetrahydrofuran and mixtures comprising two or more thereof.

In one embodiment the composition comprises water and one or other more solvents from list (iia) and/or list (iib).

Optionally the composition further comprises (iii) a monomer which is reactive with component (i), for example a monomer which comprises one polymerisable group (e.g. an ethylenically unsaturated group) and optionally one or more cationic groups. Preferred polymerisable groups are ethylenically unsaturated groups and especially (meth)acrylic groups, as described above in relation to component (i).

Preferably the number of moles of component (i) exceeds the number of moles of component (iii), when present.

The composition preferably comprises 0 to 20 wt % of component (iii).

In a preferred embodiment the amount of component (i) present in the composition relative to the total amount of components (i) and (iii) is at least 80 wt %, more preferably at least 90 wt %, especially at least 95 wt %. In the most preferred embodiment the composition comprises 0 wt % of component (iii).

Preferably component (iii) (when present) is soluble in component (ii).

The composition may be cured by any suitable process, including thermal curing, photocuring and combinations of the foregoing. However the composition is preferably cured by photocuring, e.g. by irradiating the composition and thereby causing component (i) and any other polymerisiable components present in the composition to polymerise. Typically component (ii) is inert and does not polymerise, instead leaving pores in the resultant membrane.

Preferably the composition further comprises (iv) a polymerisation initiator, e.g. a thermal initiator and/or a photoinitiator.

Examples of suitable thermal initiators which may be included in the composition include 2,2′-azobis(2-methylpropionitrile) (AIBN), 4,4′-azobis(4-cyanovaleric acid), 2,2′-azobis(2,4-dimethyl valeronitrile), 2,2′-azobis(2-methylbutyronitrile), 1,1′-azobis(cyclohexane-1-carbonitrile), 2,2′-azobis(4-methoxy-2,4-dimethyl valeronitrile), dimethyl 2,2′-azobis(2-methylpropionate), 2,2′-azobis[N-(2-propenyl)-2-methylpropionamide, 1-[(1-cyano-1-methylethyl)azo]formamide, 2,2′-Azobis(N-butyl-2-methylpropionamide), 2,2′-Azobis(N-cyclohexyl-2-methylpropionamide), 2,2′-Azobis(2-methylpropionamidine) dihydrochloride, 2,2′-Azobis[2-(2-imidazolin-2-yl)propane]dihydrochloride, 2,2′-Azobis[2-(2-imidazolin-2-yl)propane]disulfate dihydrate, 2,2′-Azobis[N-(2-carboxyethyl)-2-methylpropionamidine] hydrate, 2,2′-Azobis{2-[1-(2-hydroxyethyl)-2-imidazolin-2-yl]propane} dihydrochloride, 2,2′-Azobis[2-(2-imidazolin-2-yl)propane], 2,2′-Azobis(1-imino-1-pyrrolidino-2-ethylpropane) dihydrochloride, 2,2′-Azobis{2-methyl-N-[1,1-bis(hydroxymethyl)-2-hydroxyethl]propionamide} and 2,2′-Azobis[2-methyl-N-(2-hydroxyethyl)propionamide].

Examples of suitable photoinitiators which may be included in the composition include aromatic ketones, acylphosphine compounds, aromatic onium salt compounds, organic peroxides, thio compounds, hexaarylbiimidazole compounds, ketoxime ester compounds, borate compounds, azinium compounds, metallocene compounds, active ester compounds, compounds having a carbon halogen bond, and an alkyl amine compounds. Preferred examples of the aromatic ketones, the acylphosphine oxide compound, and the thio compound include compounds having a benzophenone skeleton or a thioxanthone skeleton described in “RADIATION CURING IN POLYMER SCIENCE AND TECHNOLOGY”, pp. 77-117 (1993). More preferred examples thereof include an alpha-thiobenzophenone compound described in JP1972-6416B (JP-S47-6416B), a benzoin ether compound described in JP1972-3981B (JP-S47-3981B), an alpha-substituted benzoin compound described in JP1972-22326B (JP-S47-22326B), a benzoin derivative described in JP1972-23664B (JP-S47-23664B), an aroylphosphonic acid ester described in JP1982-30704A (JP-S57-30704A), dialkoxybenzophenone described in JP1985-26483B (JP-S60-26483B), benzoin ethers described in JP1985-26403B (JP-S60-26403B) and JP1987-81345A (JP-S62-81345A), alpha-amino benzophenones described in JP1989-34242B (JP-H01-34242B), U.S. Pat. No. 4,318,791A, and EP0284561A1, p-di(dimethylaminobenzoyl)benzene described in JP1990-211452A (JP-H02-211452A), a thio substituted aromatic ketone described in JP1986-194062A (JP-561-194062A), an acylphosphine sulfide described in JP1990-9597B (JP-H02-9597B), an acylphosphine described in JP1990-9596B (JP-H02-9596B), thioxanthones described in JP1988-61950B (JP-S63-61950B), and coumarins described in JP1984-42864B (JP-S59-42864B). In addition, the photoinitiators described in JP2008-105379A and JP2009-114290A are also preferable. In addition, photoinitiators described in pp. 65 to 148 of “Ultraviolet Curing System” written by Kato Kiyomi (published by Research Center Co., Ltd., 1989) may be used.

The polymerisation initiator is preferably water-soluble.

The composition preferably comprises 0.1 to 5 wt %, more preferably more preferably 0.3 to 2 wt %, of the polymerisation initiator (iv). The polymerisation initiator (iv) preferably has a water-solubility of at least 1 wt %, more preferably at least 3 wt %, when measured at 25° C.

Optionally the composition includes one or more further components, e.g. a surfactant, a polymer dispersant, a polymerization reaction controlling agent, a thickening agent, an anti-crater agent, or the like, in addition to the above-described components.

Optionally the membrane of the present invention further comprises a support, especially a porous support. Inclusion of a support can provide the membrane with increased mechanical strength. If desired the composition may be applied to the support between steps (a) and (b) of the process for preparing the membranes according to the second aspect of the present invention described below. In this way the porous support may be impregnated with the composition and the composition may then be polymerised on and/or within the support.

Examples of suitable supports include synthetic woven fabrics and synthetic non-woven fabrics, sponge-like films, and films having fine through holes. The material for forming the optional porous support can be a porous membrane based on, for example, polyolefin (polyethylene, polypropylene, or the like), polyacrylonitrile, polyvinyl chloride, polyester, polyamide, or copolymers thereof, or, for example, polysulfone, polyether sulfone, polyphenylene sulfone, polyphenylene sulfide, polyimide, polyethermide, polyamide, polyamideimide, polyacrylonitrile, polycarbonate, polyacrylate, cellulose acetate, cellulose, polypropylene, poly(4-methyl-1-pentene), polyvinylidene fluoride, polytetrafluoroethylene, polyhexafluoropropylene, polychlorotrifluoroethylene, or copolymers thereof. Among these, in the present invention, polyolefin and cellulose are preferable.

As the commercially available porous support there may be used products from Japan Vilene Company, Ltd., Freudenberg Filtration Technologies, Sefar AG or Asahi-Kasei.

When the membrane comprises a support and the curing comprises photocuring then preferably the support does not shield the wavelength of light used to cure the composition.

The support is preferably a hydrophilic support, for example a support that has been subjected to a corona treatment, an ozone treatment, a sulfuric acid treatment, a silane coupling agent treatment or two or more of the foregoing treatments.

The membrane according to the present invention may optionally include more than one supports and the more than one support may be identical to each other or different.

The membrane preferably has an average pore size of 10 to 5,000 nm, more preferably 100 to 2,000 nm. Preferably the pores are deeper than their average diameter.

The average pore size of the membrane according to the present invention may be measured using a porometer, e.g. a Porolux™ porometer. For example, one may fully wet the membrane to be tested with a wetting fluid (e.g. Porefil™ wetting Fluid, an inert, non-toxic, fluorocarbon wetting fluid with zero contact angle), place the wetted membrane in the sample holder of the porometer and apply a pressure of up to 35 mbar. The porometer can then provide the bubble point, maximum pore size, mean flow pore size, minimum pore size, average pore size distribution (of uniform materials) and air permeability of the membrane under test.

When the membrane comprises a support, the membrane preferably has a porosity of 15 to 99%, preferably 20 to 99% and especially 20 to 85%.

When the membrane does not comprise a support, the membrane preferably has a porosity of 21 to 70%.

The porosity of the membrane may be determined by gas displacement pycnometry, e.g. using a pycnometer (especially the AccuPyc™ II 1340 gas displacement pycnometry system available from Micromeritics Instrument Corporation).

The porosity of the membrane is the amount of volume that can be accessed by external fluid or gas. This may be determined as described below. Preferably, the porosity of the membrane of the present invention is more than 20%.

When the membrane of the present invention includes a support, the thickness of the membrane including the support, in the dry state, is preferably 20 μm to 2,000 μm, more preferably 40 μm to 1,000 μm, and particularly preferably 70 μm to 800 μm.

When the membrane of the present invention does not comprise a support, the thickness of the membrane in a dry state is preferably 20 μm to 2,000 μm, more preferably 100 μm to 2,000 μm, and particularly preferably 150 μm to 2,000 μm.

When the membrane of the present invention includes a support, the thickness of the membrane including the support, when measured after storing for 12 hours in a 0.1 M NaCl solution, is preferably 10 μm to 4,000 μm, more preferably 20 μm to 2,000 μm and particularly preferably 20 μm to 1,500 μm.

When the membrane of the present invention does not comprise a support, the thickness of the membrane, when measured after storing for 12 hours in a 0.1 M NaCl solution, is preferably 10 μm to 4,000 μm, more preferably 50 μm to 4,000 μm and especially 70 μm to 4,000 μm.

According to a second aspect of the present invention there is provided a process for preparing a membrane according to the first aspect of the present invention comprising curing the composition defined in the first aspect of the present invention.

Preferably the membrane according to the first aspect of the present invention has been obtained by a process comprising the steps of:
(a) mixing component (i), (ii) and preferably (iii) and/or (iv) to form a composition comprising components (i), (ii) and optionally (iii);
(b) curing (e.g. irradiating) the composition arising from step (a) and thereby polymerising component (i) to form a membrane; and
(c) optionally washing the membrane arising from step (c).

Preferably the process used to prepare the membranes of the present invention comprise polymerisation-induced phase separation, more preferably photo-polymerization induced phase separation, e.g. of the membrane from the composition. In this process, preferably the polymer is formed due to a photo-polymerization reaction.

Optionally step (b) may be performed by one or more further irradiation and/or heating steps in order to fully cure the membrane.

Including component (ii) in the composition has the advantage of helping the polymerisation in step (b) proceed uniformly and smoothly.

In a preferred embodiment component (ii) acts as a solvent for component (i) and assists the formation of the pores in the resultant membrane.

The process according to the second aspect of the present invention provides substantially uniform membranes, often with a substantially uniform bicontinuous structure. In some embodiments the curing causes component (i) (and component (iii) when present) to form substantially uniform polymer particles which then merge to form the membranes of the present invention having an average pore size of 5 nm to 5,000 nm and a porosity of 15% or more. Gaps between the polymer particles provide pores of the desired average size and a membrane of the desired porosity.

The polymer particles, or agglomerates thereof, typically have an average diameter in the range of 0.1 nm to 5,000 nm. Preferably the polymer particles have an average particle or agglomerate size of 1 nm to 2,000 nm, most preferably, 10 nm to 1,000 nm. The average particle or agglomerate size may be determined by cross-sectional analysis using Scanning Electron Microscopy (SEM).

If desired the composition may be applied to a support (especially a porous support) between steps (a) and (b) of the process according to the second aspect of the present invention. Step (b) may be performed on the composition which is present on and/or in the support. When the membrane is not required to comprise a support, the membrane may be peeled-off the support. Alternatively if the membrane is required to comprise a support then the membrane may be left on and/or in the support.

The composition may be applied to the support or immersed in the support by various methods, for example, curtain coating, extrusion coating, air knife coating, slide coating, nip roll coating, forward roll coating, reverse roll coating, dip coating, kiss coating, rod bar coating, and spray coating. Coating of a plurality of layers can be performed simultaneously or sequentially. In simultaneous multilayer coating, curtain coating, slide coating, slot die coating, or extrusion coating is preferable.

The composition may be applied to a support at a temperature which assists the desired phase separation of the membrane from the composition. The temperature at which the composition is applied to the support (when present) is preferably below 80° C., more preferably between 10 and 60° C. and especially between 15 and 50° C.

When the membrane comprises a support, before the composition is applied to the surface of the support one may treat the surface of the support e.g. using a corona discharge treatment, a glow discharge treatment, a flame treatment, or an ultraviolet rays irradiation treatment. In this way one may improve the wettability and the adhesion of the support.

Step (b) optionally further comprises heating the composition.

Thus in a preferred process, the composition is applied continuously to a moving support, more preferably by means of a manufacturing unit comprising one or more composition application station(s), one or more irradiation source(s) for curing the composition, a membrane collecting station and a means for moving the support from the composition application station(s) to the irradiation source(s) and to the membrane collecting station.

The composition application station can be placed at the upstream position with respect to the irradiation source, and the irradiation source can be placed at the upstream position with respect to the composite membrane collecting station.

Preferably the curing of the composition of the present invention is initiated within 60 seconds, more preferably within 15 seconds, particularly preferably within 5 seconds and most preferably within 3 seconds from when the composition is applied to the support or from when the support has been impregnated with the composition (when a support is used).

Light irradiation for photocuring is preferably performed for less than 10 seconds, more preferably for less than 5 seconds, particularly preferably for less than 3 seconds and most preferably for less than 2 seconds. In a continuous for preparing the membrane, the membrane may be irradiated continuously. The speed at which the composition is moved through the irradiation beam created by the irradiation source then determines the cure time and radiation dose.

Preferably the composition is cured by a process comprising irradiating the composition with ultraviolet (UV) light. The wavelength of the UV light used depends on the photoinitiator present in the composition and, for example, the UV light is UV-A (400 nm to 320 nm), UV-B (320 nm to 280 nm) and/or UV-C (280 nm to 200 nm).

When high intensity UV light is used to cure the composition a significant amount of heat may be generated. In order to prevent overheating, it is preferable to cool the lamp of the light source and/or the support/membrane with cooling air. When the composition is irradiated with a high dose of infrared light (IR light) together with a UV light, irradiation with UV light is preferably performed by using an IR reflecting quartz plate as a filter.

Examples of UV light sources include a mercury arc lamp, a carbon arc lamp, a low pressure mercury lamp, a medium pressure mercury lamp, a high pressure mercury lamp, a swirling flow plasma arc lamp, a metal halide lamp, a xenon lamp, a tungsten lamp, a halogen lamp, laser, and an ultraviolet ray emitting diode. A medium pressure or high pressure mercury vapor type ultraviolet ray emitting lamp is particularly preferable. Additionally, to modify the emission spectrum of a lamp, an additive such as metal halide may be present. A lamp having an emission maximum at a wavelength of 200 nm to 450 nm is particularly suitable.

The energy output of the radiation source is preferably 20 W/cm to 1000 W/cm and more preferably 40 W/cm to 500 W/cm, but if a desired exposure dose can be achieved, the energy output may be higher or lower than the aforementioned exposure dose. By the exposure intensity, curing of the film is adjusted. The exposure dose is measured in a wavelength range of UV-A by using a High Energy UV Radiometer (UV Power Puck (Registered Trademark) manufactured by EIT-Instrument Markets), and the exposure dose is preferably 40 mJ/cm² or greater, more preferably 100 mJ/cm² to 3,000 mJ/cm², and most preferably 150 mJ/cm² to 1,500 mJ/cm². The exposure time can be freely selected, and is preferably short, and most preferably less than 2 seconds.

The membrane of the present invention is particularly useful for the separation and purification of biomolecules such as proteins, peptides, amino acids, anti-bodies and nucleic acids in biomedical applications.

The membranes of the present invention preferably have an ion-exchange capacity of 0.50 meq/g to 8.00 meq/g, more preferably 0.5 meq/g to 6.00 meq/g and especially 0.70 meq/g to 4.00 meq/g.

The ion-exchange capacity (IEC) of the membranes according to the present invention may be determined as described below.

The water flux of the membrane of the present invention is preferably more than 100 l/m²/bar/hr, more preferably more than 150 l/m²/bar/hr, especially more than 500 l/m²/bar/hr and more especially more than 1000 l/m²/bar/hr.

The water flux of the membranes according to the present invention may be determined as described below.

The swelling of the membranes of the present invention may be determined by measuring the volume of the membrane when dry and when wet and when wet with water and performing the following calculation:

$$Swelling = \frac{V o l u m {e}_{wet} - V o l u m {e}_{dry}}{V o l u m {e}_{dry}} \times 100 \%$$

The swelling of the membranes in water is preferably less than 20%, more preferable less than 10% and especially less than 5%.

According to a third aspect of the present invention there is provided use of a membrane according to the first aspect of the present invention for detecting, filtering and/or purifying biomolecules.

The membranes according to the first aspect of the present invention may be used for filtering, and/or purifying biomolecules by eluting solutions containing biomolecules, especially biomolecules which carry a negative charge. The negative charge on such biomolecules is attracted to the positive charge on the membrane derived from component (i). The membranes may be used to separate biomolecules by a number of processes, including use of the membranes in size-exclusion chromatography (e.g. where the pores of the membrane are used to separate or purify biomolecules based on their size (i.e., physical exclusion)) and in ion exchange chromatography (e.g. where biomolecules are purified or separated according to the strength of their overall ionic interaction with the cationic groups in the membrane (i.e. electronic interactions)).

The membranes according to the first aspect of the present invention may be used for detecting biomolecules by techniques involving the detection of colour, especially when the biomolecules comprise a fluorescent or colored marker.

Thus a further aspect of the present invention comprises a process for purifying a biomolecule and/or separating a biomolecule from other biomolecules comprising contacting the biomolecules with a membrane according to the present invention. Preferably the process for purifying a biomolecule and/or separating a biomolecule from other biomolecules comprises membrane size-exclusion chromatography or ion exchange chromatography.

The membranes may of course be used for other purposes too.

Preferably the membranes of the present invention are stable at pH 1.0 to pH 10.0 for at least 12 hours, more preferably for at least 16 hours.

The invention will now be illustrated by the following, non-limiting examples.

The following abbreviations are used in the Examples:
FO-2223-10 is a non-woven, polypropylene-based, porous cloth of thickness 100 μm obtained from Freudenberg Group. This acts as a support.
Novatexx™ 6320 is a non-woven, polypropylene-based, porous cloth of thickness 240 μm obtained from Freudenberg Group. This acts as a support.
Novatexx™ 2465 is a non-woven, polyethylene/polypropylene-based, porous cloth of thickness 310 μm] obtained from Freudenberg Group. This acts as a support.
Novatexx™ 2036 is a non-woven, polypropylene-based, porous cloth of thickness 230 μm obtained from Freudenberg Group. This acts as a support.
IPA is isopropanol.
(M1), (M10) and (M32) are cross-linking agents having two or one cationic groups and having the structures shown above (from FUJIFILM Corporation);
(M1) may be prepared by the method described in US20160367980, paragraph [252];
(M10) may be prepared by the method described in US20160367980, paragraph [257];
(M32) may be prepared by the following method:

To a stirred slurry of 13 grams 3-chloropropylamine hydrochloride (13 g, 100 mmol, obtained from Sigma-Aldrich) in dichloromethane (50 mL) was rapidly added a solution of acryloyl chloride (9.0 g, 100 mmol, obtained from Sigma Aldrich) in dichloromethane (50 mL). To this solution was added dropwise at 0° C. a solution of triethylamine (20.2 g, 200 mmol, obtained from Sigma-Aldrich) in dichloromethane (50 mL), and the reaction was allowed to stand at room temperature for 3 hours. The resultant precipitate was filtered-off, and the filtrate was treated with an aqueous solution (30 mL) of 8.0 grams NaOH (200 mmol) while being stirred. The aqueous layer was separated and washed twice with dichloromethane (30 mL). The combined organic phases were dried over Na₂SO₄ and then evaporated to dryness. The crude yellow oil was redissolved in a mixture of acetonitrile (156 mL), methanol (310 mL of) and 4-methoxyphenol (24.4 mg, manufactured by Tokyo Chemical Industry Co., Ltd.). Subsequently, N-[3-(dimethylamino)propyl]acrylamide (15.7 g, 100 mmol, manufactured by Tokyo Chemical Industry Co., Ltd.) was added, and the resultant product was stirred at 50° C. for 2 hours. After being allowed to react, filtration was performed, then, acetone (1.54 litres) was added thereto, followed by stirring at room temperature for 1 hour, and the produced powder was filtered, whereby 13.1 g of the compound (M32) was obtained as a white powder (yield of 89%). Characterization of (M32) was performed by ¹H NMR (62 MHz, CDCl₃, δ): 6.50-5.69 (m, 3H), 3.65 (t, 2H, J=5.58 Hz), 3.12 (t, 2H, J=5.58 Hz) and 2.00 (p, 2H, J=4.96 Hz).
CN132 is a cross-linking agent having no cationic groups and having the structure shown below (from Sartomer).
M282 is a cross-linking agent having no cationic groups and having the structure shown below (from Sigma-Aldrich).
MBA is a cross-linking agent having no cationic groups and having the structure shown below (from Sigma-Aldrich).
OXE01 is Irgacure™ Oxe01, a photoinitiator from Ciba N.V.
Irgacure™ 1173 is a photoinitiator from BASF.
DMAPAAQ is an acrylamide monomer having a cationic group (from Sigma-Aldrich) having the structure shown below.
(CL-1) is a cross-linking agent comprising two cationic groups having two cationic groups having the structure shown below. This monomer was obtained from FUJIFILM and has the structure shown below. (CL-1) may be prepared by the method described in JP2000-229917A:
(CL-2) is a cross-linking agent comprising two cationic groups having two cationic groups having the structure shown below. (CL-2) may be prepared by the method described in JP2000-229917A:

DMAPAAQ, MBA, CN132, M282, (CL-1) and (CL-2) have the structures shown below:

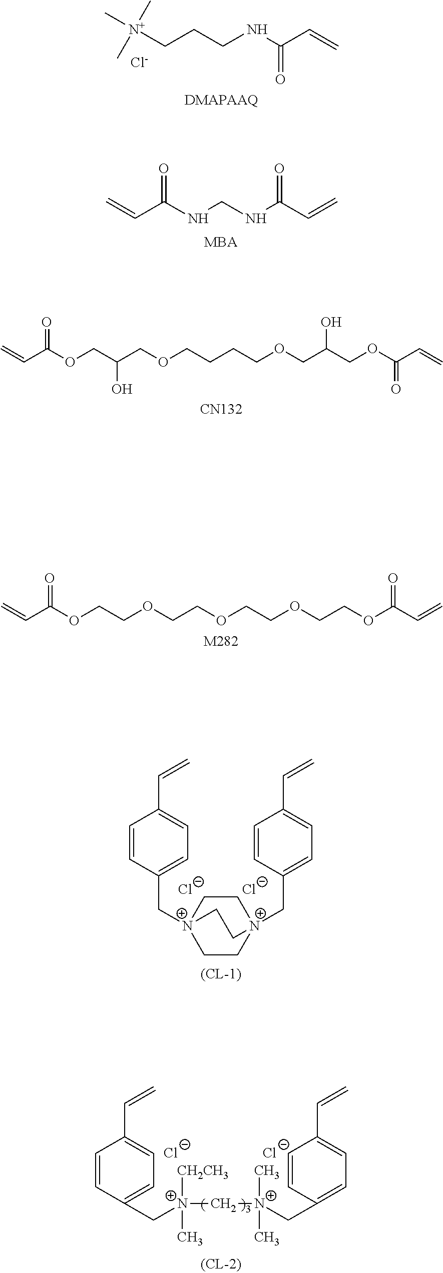

The water flux, ion-exchange capacity, porosity and thickness of the membranes described in the Examples and Comparative Example were measured as described below:

### I) Water Flux (L/m²/Bar/Hr) of the Membrane

Water flux of the membranes was measured using a device where the weight of water passing through the membrane was measured over time. A column of feed solution (pure water) was brought into contact with the membrane under evaluation and the feed solution was forced through the membrane by a constant applied air pressure on top of the water column. By achieving a constant flow of water at a constant applied pressure, the water flux could be determined.

Typically the membrane under evaluation was stored for 12 hours in pure water prior to use. The feed solution (250 ml of pure water) was brought into contact with the membrane (film contact area of 12.19 cm²). The water column was closed and pressurized with air pressure and the membrane was flushed with one water column (250 ml). The feed solution was refreshed and a constant air pressure of 100 mbar was applied. Finally, the measurements were performed by monitoring the weight by balance at a constant flow.

### II) Ion-Exchange Capacity (Meq/g) of the Membrane (“IEC”)

Prior to measuring a membrane's IEC, the membrane was weighed in the dry state. The membrane was then stored in 1.0 M KCl solution for 24 hours to fully exchange all possible counter-ions of the membrane for chloride ions and then the membrane was stored in demineralised water for 24 hours. Subsequently, the membrane was equilibrated with 0.1 M KBr solution for 24 hours and rinsed with demineralised water for 24 hours. The remainder of KBr solution and the rinsing solution of demineralised water were combined quantitatively; 1.0 g of Barium acetate was added and the solution was titrated with 0.1 M AgNO₃. The amount of silver ions were measured using an ion-selective silver electrode, which resulted in an amount of ions which had been exchanged per unit weight of membrane.

### III) Porosity (%) of the Membrane

The porosity of the membrane under evaluation was determined from the apparent density (ρₐₚₚₐᵣₑₙₜ) and the real density of the membrane. The ρₐₚₚₐᵣₑₙₜ was measured in air by weighing the membrane and determining its volume from the dimensions of the membrane (length, width and thickness). The real density of the membrane was determined from pycnometer measurements of the membrane with known weight under helium atmosphere. The Helium occupied the pores of the membrane with known weight, and therefore the volume of polymer could be determined. From this the porosity could be determined according to Formula (1):

$$Porosity = \frac{{ρ}_{real} - {ρ}_{apparent}}{{ρ}_{real}} \times 100 \% \qquad ( 1 )$$

The pycnometer used was the AccuPyc™ II 1340 gas displacement pycnometry system from Micromeritics Instrument Corporation.

### IV) Thickness (μm) of the Membrane

The thickness of the membranes was determined by contact mode measurement. The measurements were performed at five different positions of the membrane and the average thickness of these five measurements in μm was calculated.

### V) pH Stability of the Membrane by Water Flux Measurement

The pH stability of the membrane was determined by measuring the water flux of the membrane before and after exposing the membrane to aqueous solutions of pH 1, 3, 8 and 10 for 16 hours.

The membranes under evaluation were stored for 12-16 hours in pure water (pH 5.5) before their pH stability was measured. A column of pure water was closed and pressurized with air pressure and the membrane was flushed with one water column (250 ml). Then another column of pure water was used as feed solution for the water flux measurement (as described above). The feed solution was brought into contact with the membrane (film contact area of 12.19 cm²) and a constant air pressure of 100 mbar was applied. Finally, the flux measurements of the membrane, before challenging the membrane for a different pH, were performed by monitoring the weight of the filtrate by balance at a constant flow. The membranes were removed from the setup and stored for 16 hours in aqueous hydrochloric acid (when assessing membrane stability at pH 1 and pH 3) or aqueous NaOH (when assessing membrane stability at pH 8 and pH 10). The membranes under evaluation were removed from the challenging solution and placed back in the setup. A feed solution (250 ml column of pure water) was brought into contact with the membrane. The water column was closed and pressurized with air pressure (100 mbar) and the membrane was flushed with one water column (250 ml). The feed solution was refreshed and a constant air pressure of 100 mbar was applied. Finally, the measurements after exposure to a challenging pH solution were performed by monitoring the weight of the filtrate by balance at a constant flow. Membranes having a difference in water flux of less than 10% before and after exposure for 16 hours to aqueous solutions at all of pH 1, 3, 8 and 10 were deemed to have good pH tolerance. Membranes having a difference in water flux of 10% or more before and after exposure for 16 hours to aqueous solutions of pH 1, pH 3, pH 8 and/or pH 10 were deemed to have a bad pH tolerance.

### EXAMPLES 1 TO 31

### (ai) Preparation of Compositions

Compositions 1 to 31 were prepared by mixing the ingredients indicated in Table 1 below in the specified amounts. In Table 1, component (i) had the structure identified above in the description, component (ii) was as described in Table 1 and component (iv) was Irgacure™ 1173. The compositions were each applied to the support indicated in Table 1 as described in more detail further on in this specification.

TABLE 1
Compositions

| Composition | | Amount of | Component (ii) | | | |
|---|---|---|---|---|---|---|
| and | Component | Component | water | IPA | Component | |
| Example | (i) | (i) (wt %) | (wt %) | (wt %) | (iv) (wt %) | Support |
| 1 | (M10) | 24.23 | 14.23 | 61.38 | 0.15 | FO-2223-10 |
| 2 | (M10) | 24.23 | 14.23 | 61.38 | 0.15 | Novatexx ™ |
| | | | | | | 6320 |
| 3 | (M10) | 24.23 | 14.23 | 61.38 | 0.15 | Novatexx ™ |
| | | | | | | 2465 |
| 4 | (M10) | 24.23 | 14.23 | 61.38 | 0.15 | Novatexx ™ |
| | | | | | | 2036 |
| 5 | (M10) | 25.82 | 15.16 | 58.85 | 0.16 | FO-2223-10 |
| 6 | (M10) | 26.69 | 15.68 | 57.46 | 0.17 | FO-2223-10 |
| 7 | (M10) | 27.63 | 16.23 | 55.96 | 0.18 | FO-2223-10 |
| 8 | (M10) | 28.43 | 16.70 | 54.69 | 0.18 | FO-2223-10 |
| 9 | (M10) | 30.29 | 17.79 | 51.73 | 0.19 | FO-2223-10 |
| 10 | (M10) | 30.88 | 18.14 | 50.78 | 0.20 | FO-2223-10 |
| 11 | (M10) | 33.51 | 19.68 | 46.60 | 0.21 | FO-2223-10 |
| 12 | (M10) | 35.80 | 21.02 | 42.95 | 0.23 | FO-2223-10 |
| 13 | (M10) | 39.38 | 23.13 | 37.25 | 0.25 | FO-2223-10 |
| 14 | (M1) | 37.91 | 19.53 | 42.33 | 0.23 | FO-2223-10 |
| 15 | (M1) | 37.33 | 19.23 | 43.21 | 0.23 | FO-2223-10 |
| 16 | (M1) | 35.69 | 22.79 | 41.31 | 0.22 | FO-2223-10 |
| 17 | (M1) | 34.18 | 26.04 | 39.56 | 0.21 | FO-2223-10 |
| 18 | (M1) | 31.52 | 31.80 | 36.49 | 0.19 | FO-2223-10 |
| 19 | (M1) | 29.25 | 36.72 | 33.85 | 0.18 | FO-2223-10 |
| 20 | (M1) | 38.86 | 20.02 | 40.91 | 0.22 | FO-2223-10 |
| 21 | (M1) | 41.03 | 21.14 | 37.63 | 0.20 | FO-2223-10 |
| 22 | (M1) | 42.89 | 22.09 | 34.83 | 0.19 | FO-2223-10 |
| 23 | (M1) | 34.81 | 17.93 | 47.04 | 0.21 | FO-2223-10 |
| 24 | (M1) | 33.14 | 17.07 | 49.59 | 0.20 | FO-2223-10 |
| 25 | (M1) | 31.62 | 16.29 | 51.90 | 0.20 | FO-2223-10 |
| 26 | (M1) | 30.23 | 15.57 | 54.02 | 0.19 | FO-2223-10 |
| 27 | (M1) | 28.96 | 14.92 | 55.95 | 0.18 | FO-2223-10 |
| 28 | (M1) | 34.49 | 22.03 | 43.27 | 0.21 | FO-2223-10 |
| 29 | (M1) | 32.06 | 24.43 | 43.32 | 0.20 | FO-2223-10 |
| 30 | (M1) | 28.09 | 28.34 | 43.40 | 0.17 | FO-2223-10 |
| 31 | (M32) | 24.23 | 14.23 | 61.38 | 0.15 | FO-2223-10 |

(aii) Application of the Compositions to a Support

The compositions described in Table 1 above were each independently applied to the supports indicated in Table 1 at 20° C. using a tabletop coating machine (manufactured by TQC, Model AB3000 Automatic film applicator). The supports were attached to an aluminium plate and the compositions were applied to the supports at a speed of about 1 cm/sec using a wire bar (a stainless steel bar on which a wire of 150 μm had been wound at 1 lap/3 cm (length direction). Any excess composition was removed from the coated supports using a 12 μm wire bar. A sheet of polyethylene was placed on top of the coated support and any air bubbles present in the coating composition were removed by applying the 12 μm wire bar to the sheet of polypropylene.

### (b) Curing the Compositions to Form the Membrane

The top polypropylene sheet used to remove bubbles was removed from the coated supports prepared in step (aii) and the compositions present on the supports were cured by irradiation with UV using a Light Hammer LH6 UV exposure machine (manufactured by Fusion UV Systems, Inc.). The Light Hammer machine was fitted with a Model H-bulb (100% strength) and a D-bulb (80% strength). The coated supports were passed through the Light Hammer machine at a speed of 10 m/min to expose the composition to the UV light from both bulbs. The curing time was 0.8 seconds. The exposure time was twice 0.71 seconds. The first bulb performed most of the curing and the second bulb provided additional curing, improving the mechanical strength of the resultant membrane. The resultant membrane was removed from the aluminium plate and was stored in a polyolefin bag.

The membrane resulting from Example 1, Step (b), had a dry thickness of 118 μm, an ion-exchange capacity of 1.42 meq/g, a water flux of 2400 l/m²/bar/hr and a swelling in water of 2.3%.

### Properties of the Resultant Membranes

The membranes obtained in Step (b) above had the properties described in Table 2 below:

TABLE 2
Membrane Properties

| | | | | Dry | Wet | | Average |
|---|---|---|---|---|---|---|---|
| | Waterflux | IEC | Porosity | thickness | thickness | Swelling | Pore Size |
| Example | (l/m²/bar/hr) | (meq/g) | (%) | (μm) | (μm) | (%) | (nm) |
| 1 | 2400 | 1.42 | 46 | 118 | 119 | 2.3 | 610 |
| 2 | 1669 | 1.82 | 68 | 222 | 223 | 1.5 | 598 |
| 3 | 1448 | 1.84 | 66 | 244 | 245 | 1.0 | 578 |
| 4 | 8877 | 1.73 | 63 | 211 | 212 | 2.5 | 806 |
| 5 | 445 | ND | 44 | 96 | 97 | 0.5 | 210 |
| 6 | 509 | ND | 43 | 100 | 101 | 0.6 | 215 |
| 7 | 208 | ND | 42 | 112 | 113 | 0.6 | 185 |
| 8 | 211 | ND | 42 | 98 | 99 | 0.6 | 113 |
| 9 | 155 | ND | 40 | 109 | 110 | 0.6 | 125 |
| 10 | 860 | ND | 39 | 102 | 103 | 0.5 | 234 |
| 11 | 268 | ND | 36 | 105 | 106 | 0.6 | 101 |
| 12 | 255 | ND | 34 | 95 | 96 | 0.6 | 109 |
| 13 | 268 | ND | 31 | 110 | 111 | 0.6 | 98 |
| 14 | 202 | 1.12 | 32 | 108 | 109 | 0.5 | 109 |
| 15 | 411.09 | 1.53 | 37 | 100 | 101 | 0.6 | 208 |
| 16 | 244.57 | ND | 38 | 112 | 112 | 0.5 | 87 |
| 17 | 1942.47 | ND | 39 | 107 | 108 | 0.7 | 555 |
| 18 | 606.09 | ND | 41 | 107 | 108 | 0.7 | 252 |
| 19 | 1860.14 | 1.53 | 43 | 110 | 111 | 0.8 | 602 |
| 20 | ND | 1.53 | 35 | 102 | 103 | 1.0 | ND |
| 21 | 3330.17 | ND | 33 | 103 | 103 | 0.5 | 757 |
| 22 | ND | 1.55 | 31 | 109 | 109 | 0.5 | ND |
| 23 | 550.12 | 1.56 | 39 | 105 | 105 | 0.5 | 251 |
| 24 | 241.62 | 1.80 | 41 | 102 | 102 | 0.5 | 122 |
| 25 | 185.56 | 1.75 | 42 | 113 | 113 | 0.5 | 112 |
| 26 | 461.82 | 1.61 | 44 | 108 | 109 | 0.6 | 229 |
| 27 | 398.04 | 1.57 | 45 | 101 | 102 | 0.6 | 210 |
| 28 | 2173.82 | 1.52 | 39 | 112 | 113 | 0.6 | 599 |
| 29 | 369.67 | 1.57 | 41 | 106 | 107 | 0.6 | 189 |
| 30 | 401.22 | 1.57 | 45 | 105 | 106 | 0.6 | 214 |
| 31 | 2500 | 0.85 | 46 | 118 | 119 | 2.3 | 625 |

ND means not determined.

### EXAMPLE 32

In this Example a membrane was prepared which did not comprise a support. Furthermore, the membrane was cured thermally instead of photocuring.

### Preparation of Composition 32

Composition 32 was prepared my mixing the ingredients indicated in Table 3 in the specified amounts. In Table 3, component (i) had the structure identified above in the description, component (ii) was water and isopropanol. The compositions were each applied to the support indicated in Table 1 as described above in relation to Examples 1 to 31.

TABLE 3
Composition 32

| | | Amount of | Component (ii) | | | Amount of |
|---|---|---|---|---|---|---|
| | Component | Component | Water | IPA | Component | Component |
| Composition | (i) | (i) (wt %) | (wt %) | (wt %) | (iv) | (iv) (wt %) |
| 32 | ((M10)) | 30.29 | 17.79 | 51.66 | 4,4′-azobis(4- | 0.26 |
| | | | | | cyanovaleric | |
| | | | | | acid) | |

Composition 32 (5.0 cm³) was placed in a glass vial having a capacity of 25 cm³. The vial was sealed and placed in a vacuum oven at 45° C. for 60 min. The oven was cooled down to room temperature and the vial was removed from the oven. The resultant membrane was removed from the vial and was found to have a dry thickness of 310 μm, an ion-exchange capacity of 1.42 meq/g, a water flux of 20001/m²/bar/hr and a swelling in water of 7.9%.

### Measurement of pH Tolerance

The pH tolerances of the membranes indicated in Table 4 below were measured by the method described above and the results (good or bad) are shown in the final column of Table 4:

TABLE 4
pH tolerance

| | Water flux | | | | | |
|---|---|---|---|---|---|---|
| | before | Water flux | Water flux | Water flux | Water flux | |
| Example/ | (pH 5.5) | after pH 1 | after pH 3 | after pH 8 | after pH 10 | pH |
| composition | (l/m²/bar/hr) | (l/m²/bar/hr) | (l/m²/bar/hr) | (l/m²/bar/hr) | (l/m²/bar/hr) | tolerance |
| 1 | 2400 | 2557 | 2484 | 2510 | 2588 | good |
| 10 | 860 | 939 | 862 | 902 | 925 | good |
| 26 | 462 | 488 | 465 | 471 | 507 | good |
| 30 | 401 | 426 | 412 | 410 | 439 | good |
| 31 | 2500 | 2587 | 2506 | 2593 | 2609 | good |

### COMPARATIVE EXAMPLES CEX1 TO CEX11

### Preparation of Compositions

Comparative composition CEx1 to CEx11 were prepared by mixing the ingredients indicated in Table 5 below in the specified amounts. In Table 5, component (i) had the structure identified above in the description, component (ii) was water and isopropanol. In CEx1 to CEx9 Component (iv) was Irgacure™ 1173 and in CEx10 and CEx11 component (iv) was OXE01 and Irgacure™ 1173 in the amounts indicated.

The membranes of Comparative Examples CEx1 to CEX11 were prepared using the method described for Examples 1 to 31 above, steps (aii) and (b), except that the compositions indicated in Table 5 below were used. The support used in all cases was FO-2223-10.

TABLE 5
Compositions Used in the Comparative Examples

| | | Cross- | | Mono- | | | |
|---|---|---|---|---|---|---|---|
| Composition | | linking | | functional | Component (ii) | | |
| and | Cross- | agent | Mono- | monomer | | Other inert | Component |
| Comparative | linking | amount | functional | amount | Water | solvent | (iv) amount |
| Example | agent | (wt %) | monomer | (wt %) | (wt %) | (wt %) | (wt %) |
| CEx1 | (M1) | 65.83 | None | 0.00 | 33.91 | 0.00 | 0.26 |
| CEx2 | (M10) | 65.83 | None | 0.00 | 33.91 | 0.00 | 0.26 |
| CEx3 | (M32) | 65.83 | None | 0.00 | 33.91 | 0.00 | 0.26 |
| CEx4 | CN132 | 37.50 | None | 0.00 | 49.0 | IPA (12.50) | 1.00 |
| CEx5 | CN132 | 27.50 | DMAPAAQ | 10.00 | 49.0 | IPA (12.5 | 1.00 |
| CEx6 | M282 | 33.00 | None | 0.00 | 49.0 | IPA (17.0 | 1.00 |
| CEx7 | M282 | 27.50 | DMAPAAQ | 10.00 | 49.0 | IPA (12.5 | 1.00 |
| CEx8 | MBA | 1.30 | DMAPAAQ | 11.25 | 14.36 | DMF (9.57) | 0.11 |
| | | | | | | Dioxane | |
| | | | | | | (61.77) | |
| | | | | | | Glycerol | |
| | | | | | | (1.69) | |
| CEx9 | M282 | 12.50 | DMAPAAQ | 25.00 | 49.0 | 12.5 | 1.00 |
| CEx10 | (CL-1) | 69.0 | None | 0.00 | 20.0 | 10.0 | OXE01 (0.5) |
| | | | | | | | 1173 (0.5) |
| CEx11 | (CL-2) | 69.0 | None | 0.00 | 20.0 | 10.0 | OXE01 (0.5) |
| | | | | | | | 1173 (0.5) |

### Properties of the Resultant Comparative Membranes

The membranes obtained from Comparative Compositions CEx1 to CEx11 had the properties described in Table 6 below:

TABLE 6
Properties of the Comparative Membranes

| | | | | | | | Average | |
|---|---|---|---|---|---|---|---|---|
| | | | | Dry | Wet | | Pore | |
| | Waterflux | IEC | Porosity | thickness | thickness | Swelling | size | pH |
| Example | (l/m²/bar/hr) | (meq/g) | (%) | (μm) | (μm) | (%) | (nm) | tolerance |
| CEx1 | 74.20 | 1.43 | 9.6 | 115 | 115 | 0.5 | 4 | Good |
| CEx2 | <70 | 1.42 | 11 | 101 | 101 | 0.5 | 3 | Good |
| CEx3 | <70 | 0.75 | 8.5 | 105 | 105 | 0.5 | 1 | Good |
| CEx4 | 4000 | 0.00 | 32 | 100 | 100 | 0.5 | 200 | Bad |
| CEx5 | 150 | 0.30 | 32 | 112 | 134 | 20 | 300 | Bad |
| CEx6 | 100 | 0.00 | 37 | 116 | 116 | 0.5 | 200 | Bad |
| CEx7 | 150 | 0.30 | 37 | 116 | 139 | 20 | 300 | Bad |
| CEx8 | 10000 | 0.20 | 57 | 98 | 176 | 80 | 1000 | Bad |
| CEx9 | 100 | 0.75 | 37 | 113 | 170 | 50 | 4 | Bad |
| CEx10 | 0.0022 | ND | ND* | 103 | ND | ND | <1.0 | Good |
| CEx11 | 0.0020 | ND | ND* | 108 | ND | ND | <1.0 | Good |

ND means not determined.
*the polymers arising from CEx10 and CEx11 were extremely dense and non-porous.

In CEx1 to CEx3 component (ii) was present in less than the amount required by the present invention. The resultant membranes had very small pores and suffered from low porosity and low water flux.

In CEx4 component (i) was omitted. Therefore CEx4 contained less than 5 wt % of component (i). CEx4 had no cationic groups.

In CEx5, CEx7 to CEx9 component (i) was omitted. Instead cationic groups were provided in the membrane by the presence of DMAPAAQ. The resultant membranes suffered from low IEC.

CEx10 and CEx11 contained more than 64 wt % of component (i) and less than 36 wt % of component (ii). The resultant membranes were highly dense and had an extremely low waterflux.

1. A membrane having an average pore size of 5 nm to 5,000 nm and a porosity of 15% or more, said membrane being obtained by a process comprising curing a composition comprising:
5 to 64 wt % of (i) a cross-linking agent comprising at least one cationic group; and
36 to 95 wt % of (ii) inert solvent(s).

2. The membrane according to claim 1 wherein component (i) comprises at least two cationic groups.

3. The membrane according to claim 1 wherein component (ii) comprises water and an inert, water-miscible organic solvent wherein the wt % of water is lower than the wt % of the inert, water-miscible organic solvent.

4. (canceled)

5. The membrane according to claim 1 wherein: component (ii) comprises a composition comprising one of inert solvents selected from list (iia) and one or more inert solvents selected from list (iib):
list (iia): iso-propanol, methanol, ethanol, acetone, tetramethyl urea, hexamethyl phosphoramide, hexamethyl phosphorotriamide, butanone, cyclohexanone, methylethylketone, tetrahydrofuran, tetrahydropyran, 2-methyltetrahydrofuran, cyclopentylmethylether, propionitrile, acetonitrile, 1,4-dioxane, 1,3-dioxolane, ethyl acetate, y-butyrolactone or a mixture comprising two or more thereof; and
list (iib): water, glycerol, ethylene glycol, dimethyl sulfoxide, sulpholane, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate and y-butyrolactone, and among these,
dimethyl sulfoxide, N-methyl pyrrolidone, N,N-dimethyl formamide, dimethyl imidazolidinone, N-methyl morpholine, acetone, cyclopentylmethylether, methylethylketone, acetonitrile, tetrahydrofuran, 2-methyltetrahydrofuran and mixtures comprising two or more thereof.

6. The membrane according to claim 1 wherein component (ii) comprises isopropanol and water.

7. The membrane according to claim 5 wherein the component (ii) comprises 40 to 70 wt % of inert solvent(s) selected from list (iia) and 10 to 40 wt % of inert solvent(s) selected from list (iib).

8. The membrane according to claim 1 wherein the component (ii) comprises 40 to 70 wt % of isopropanol and 10 to 40 wt % of water.

9. The membrane according to claim 1 wherein the composition further comprises 0 to 20 wt % of (iii) a monomer which is reactive with component (i).

10. The membrane according to claim 1 wherein the curing comprises photocuring.

11. The membrane according to claim 1 wherein the composition further comprises a photoinitiator.

12. The membrane according to claim 1 wherein the photocuring comprises polymerisation-induced phase separation of the membrane from the composition.

13. The membrane according to claim 1 which further comprises a porous support.

14. (canceled)

15. A process for preparing a membrane according to claim 1, said process comprising curing a composition comprising:
5 to 64 wt % of (i) a cross-linking agent comprising at least one cationic group; and
36 o 95 wt % of (ii) inert solvent(s).

16. (canceled)

17. The process according to claim 15 wherein the curing comprises photocuring.

18. The process according claim 15 wherein the curing comprises photo-polymerization induced phase separation of the membrane from the composition.

19. (canceled)

20. A process for purifying a biomolecule and/or separating a biomolecule from other biomolecules comprising contacting the biomolecules with a membrane according to claim 1.

21. (canceled)

22. The membrane according to claim 1 wherein the composition comprises:
5 to 64 wt % of (i) a cross-linking agent comprising at least one cationic group;
36 to 95 wt % of (ii) inert solvent(s);
0 to 20 wt % of (iii) a monomer which is reactive with component (i); and
0.1 to 5 wt % of (iv) a polymerisation initiator;
wherein:
component (ii) comprises a composition comprising one of the inert solvents selected from list (iia) and one or more inert solvents selected from list (iib):
list (iia): iso-propanol, methanol, ethanol, acetone, tetramethyl urea, hexamethyl phosphoramide, hexamethyl phosphorotriamide, butanone, cyclohexanone, methylethylketone, tetrahydrofuran, tetrahydropyran, 2-methyltetrahydrofuran, cyclopentylmethylether, propionitrile, acetonitrile, 1,4-dioxane, 1,3-dioxolane, ethyl acetate, y-butyrolactone or a mixture comprising two or more thereof; and
list (iib): water, glycerol, ethylene glycol, dimethyl sulfoxide, sulpholane, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate and y-butyrolactone, and among these, dimethyl sulfoxide, N-methyl pyrrolidone, N,N-dimethyl formamide, dimethyl imidazolidinone, N-methyl morpholine, acetone, cyclopentylmethylether, methylethylketone, acetonitrile, tetrahydrofuran, 2-methyltetrahydrofuran and mixtures comprising two or more thereof.

23. The membrane according to claim 1 which is suitable for detecting, filtering and/or purifying biomolecules.

24. The process according to claim 15 wherein the composition comprises:
5 to 64 wt % of (i) a cross-linking agent comprising at least one cationic group;
36 to 95 wt % of (ii) inert solvent(s);
0 to 20 wt % of (iii) a monomer which is reactive with component (i); and
0.1 to 5 wt % of (iv) a polymerisation initiator;
wherein:
component (ii) comprises a composition comprising one of the inert solvents selected from list (iia) and one or more inert solvents selected from list (iib):
list (iia): iso-propanol, methanol, ethanol, acetone, tetramethyl urea, hexamethyl phosphoramide, hexamethyl phosphorotriamide, butanone, cyclohexanone, methylethylketone, tetrahydrofuran, tetrahydropyran, 2-methyltetrahydrofuran, cyclopentylmethylether, propionitrile, acetonitrile, 1,4-dioxane, 1,3-dioxolane, ethyl acetate, y-butyrolactone or a mixture comprising two or more thereof; and
list (iib): water, glycerol, ethylene glycol, dimethyl sulfoxide, sulpholane, dimethyl imidazolidinone, sulfolane, N-methyl pyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate and y-butyrolactone, and among these, dimethyl sulfoxide, N-methyl pyrrolidone, N,N-dimethyl formamide, dimethyl imidazolidinone, N-methyl morpholine, acetone, cyclopentylmethylether, methylethylketone, acetonitrile, tetrahydrofuran, 2-methyltetrahydrofuran and mixtures comprising two or more thereof.

25. The process according to claim 24 wherein the curing comprises photo-polymerization induced phase separation of the membrane from the composition.
